(19) United States
(12) Patent Application Publication
(10) Pub. No.: US 2022/0008110 A1
(43) Pub. Date: Jan. 13, 2022

(54) METHODS OF TREATING SUBCUTANEOUS FAT LAYERS

(71) Applicant: MIRAKI INNOVATION THINK TANK LLC, Cambridge, MA (US)

(72) Inventors: Christopher VELIS, Lexington, MA (US); Karen MILLER, South Dartmouth, MA (US); Tarik S. CHAUDHRY, Boston, MA (US); Emilia JAVORSKY, Watertown, MA (US); Rainuka GUPTA, Newton, MA (US)

(73) Assignee: MIRAKI INNOVATION THINK TANK LLC, Cambridge, MA (US)

(21) Appl. No.: 17/281,802

(22) PCT Filed: Oct. 4, 2019
(86) PCT No.: PCT/US2019/054834

§ 371 Date: Mar. 31, 2021

Related U.S. Application Data
(60) Provisional application No. 62/741,286, filed on Oct. 4, 2018.

(51) Int. Cl. A61B 18/02

(52) U.S. Cl. CPC A61B 18/02; A61B 2018/00464; A61B 2018/0293

(57) ABSTRACT

A slurry is injected into a subject at a treatment site selected from the group consisting of (i) a deep subcutaneous fat layer, (ii) a superficial subcutaneous fat layer, and (iii) the deep subcutaneous fat layer and the superficial subcutaneous fat layer. Fat cells in the selected treatment site are ablated by the slurry. For example, the slurry is injected into (i) the deep subcutaneous fat layer only, (ii) the superficial subcutaneous fat layer only, (iii) the deep subcutaneous fat layer followed by the superficial subcutaneous fat layer, (iv) the superficial subcutaneous fat layer followed by the deep subcutaneous fat layer, or (v) the deep subcutaneous fat layer and the superficial subcutaneous fat layer simultaneously.

### TECHNICAL FIELD

The invention is directed to methods of treatment and removal of subcutaneous fat by selectively targeting subcutaneous fat layers and components thereof.

### BACKGROUND

In humans, subcutaneous fat is found just beneath the skin and acts as padding and as an energy reserve in addition to providing minor thermoregulation from insulation. However, subcutaneous fat has been shown to play a role in metabolic dysfunction and systemic inflammation in human subjects. Excess subcutaneous fat, or subcutaneous adipose tissue, leads to serious health and cosmetic issues. Some health consequences of excess adipose tissue, such as type II diabetes and cardiovascular disease are associated with decreased life expectancy.

Subcutaneous adipose tissue is composed of adipocytes (fat cells) grouped together in lobules separated by connective tissues and is not homogeneous across all body areas. The size of adipocytes varies according to the nutritional state of the body, and the biology of adipocytes varies among different areas of the body. In some areas of the body, such as the torso, the subcutaneous adipose tissue is divided into two layers separated by a fascial plane. The upper layer is called the “superficial subcutaneous adipose tissue” (sSAT). The sSAT is characterized by a lamellar pattern having regular, defined cuboid fat lobules tightly organized within vertically oriented fibrous septae. The lower layer is called the “deep subcutaneous adipose tissue” (dSAT). The dSAT is characterized by a loose areolar pattern and has fat lobules are flat shaped, irregular in size, and are surrounded by high amounts of loose connective tissue. Both sSAT and dSAT layers also comprise sublayers.

One method of treating health and cosmetic issues resulting from excess subcutaneous fat is removal of the excess subcutaneous fat. Conventional non-invasive and minimally-invasive fat removal modalities such as topical cryolipolysis and other energy-based therapies, such as topically applied laser, radiofrequency, and ultrasound are limited by depth, and are only able to target sSAT.

Another method of treatment is liposuction, which is invasive and uses a cannula and suction to remove fat. The dSAT is the main target of liposuction. Because the dSAT has a loose density compared to the lamellar density of the sSAT, the dSAT is easier to remove using suction through a cannula. Removal of dSAT can allow for a more dramatic cosmetic and aesthetic improvement. Further, the dSAT has an overlying layer of sSAT to blunt the appearance of any irregularities and is more forgiving cosmetically than sSAT. Removal of fat has very little margin for error in areas of the body that have an sSAT layer but not a dSAT layer. Any minor irregularities in fat removal in the sSAT layer result in contour deformities in overlying skin, thereby contributing to a poor aesthetic result.

### SUMMARY

The present invention provides minimally invasive methods for fat removal by injecting a slurry into a deep subcutaneous fat layer, a superficial subcutaneous fat layer, or both the deep subcutaneous fat layer and the superficial subcutaneous fat layer. The slurry of the present invention can be used in selective injection cryolipolysis for fat removal, selective targeting of non-adipocyte, lipid rich tissue, and connective tissue remodeling, while avoiding non-specific hypertonic injury to tissue. Unlike conventional approaches, methods of the invention allow for selection of the treatment site and subsequent cryolipolysis, or cell death by freezing, of the underlying deep subcutaneous fat and/or the superficial subcutaneous fat. Therefore, the invention allows for selective targeting of a particular treatment site in a subject for removal of fat cells at the treatment site.

The deep subcutaneous fat layer is not accessible using topical applications of cryolipolysis, but the present invention allows selection of dSAT as a treatment site. dSAT may contribute to excess adiposity, and it also plays an important metabolic and inflammatory role. Treating dSAT with a slurry allows for treatment of the subcutaneous fat layer that is distinct from the superficial subcutaneous fat layer both in form and function. Because deep fat is anatomically and functionally distinct from superficial fat, targeting and removal of fat cells using a slurry allows for improved cosmetic outcomes and/or treatment of medical conditions associated with deep fat expansion.

The invention allows use of slurries for a highly selective, precise, and targeted removal of fat in both subcutaneous fat layers, resulting in superior results as compared to conventional methods. The invention allows for injection into sSAT only, injection into dSAT only, injection into dSAT followed by injection into sSAT, injection into sSAT followed by injection into dSAT, and injection into dSAT and sSAT simultaneously. Therefore, the invention allows for selective targeting of sSAT and dSAT.

Additionally, within each subcutaneous layer of fat there exist sublayers separated by fascia and compartments separated by fibrous tissue such as fibrous septae or connective tissue. The invention allows for use of slurries for highly selective, precise, and targeted removal of fat within individual sublayers or compartments. Further, the slurry allows for the disruption of fibrous tissue in order to simultaneously target multiple compartments within a layer of subcutaneous fat.

The slurry may be injected by any suitable means, such as injection by a cannula such as a needle. Delivery devices that may be utilized for injecting the slurry are disclosed, for example, in International Application Publication No. PCT/US2017/048995 and U.S. Provisional Application No. 62/381,231, which are incorporated herein by reference in their entirety. In some embodiments, the slurry is injected into superficial subcutaneous fat, and then the needle is moved deeper in the deep subcutaneous fat regions. Further, a slurry for use in the invention may also be injected at multiple sites selectively in either the superficial fat layer, the deep fat layer, or both. For example, the injection sites may form a pattern, such as a grid-like pattern. In another example, one injection site is used repeatedly, thereby reducing the number of injection sites and concomitant scarring potential. Each injection site is the site of a single puncture by, for example, a needle. Treatment of the patient comprises the totality of the injection and deposition sites.

A slurry for use in the invention may comprise liquid water, ice and one or more additives. For example, the slurry ice coefficient (defined as the percentage of ice particles in a slurry) can be in a range from about 2% to about 70%. A slurry used in methods of the invention may be applied at a temperature from about −25° C. to about 10° C.

Slurries for use in the invention may be any suitable composition capable of removing adipose cells. Preferably, slurries used in the invention are safe and effective for injection in humans. In some embodiments, the of the one or more additives comprise one or more of a salt, sugar and a thickener. Examples include sodium chloride, glycerol, glycerin, polyethylene glycol, dextrose, xanthan gum, and sodium carboxymethylcellulose (CMC). In an embodiment, the slurry comprises liquid water, ice particles, and an agent affecting the tonicity. Examples of additives affecting tonicity include salts, cations, anions, sugars, and sugar alcohols. In some embodiments, the osmolality of the slurry is less than about 2,200 milli-Osmoles/kilogram. In some embodiments, the osmolality of the slurry is less than about 600 milli-Osmoles/kilogram.

Any suitable amount of slurry that is safe for administering to the human subject may be injected. In an embodiment, an amount of slurry injected comprises about 60 ml or less per injection site. In some examples, the amount of slurry injected is about 1 ml to about 60 ml per injection site. In an embodiment, the amount of slurry injected comprises about 2 L or less per injection site. In some examples, the amount of slurry injected is about 1 ml to about 2 L per injection site. Different patients have different amounts of subcutaneous fat. Therefore, some patients may require injection of greater amounts of slurry in order to produce visible effects of reduction and removal of subcutaneous fat. Other patients may require multiple treatments to produce effects.

Treatment with the slurry comprises reduction or removal of fat cells in the human subject by freezing, or cryolipolysis. However, treatment may also comprise tightening of skin of the human subject. The tightening of the skin results from a collagen response upon removal and reduction of the fat cells in the subcutaneous fat layer. Reduction of subcutaneous fat may also reduce adipose tissue hypoxia or inflammatory signaling in overweight and obese individuals. Additionally, the slurry may also be utilized to mechanically disrupt fibrous tissue between compartments of subcutaneous fat, allowing the subcutaneous fat to spread and create a visually smoother appearance, for example in the treatment of cellulite.

Methods of the invention further comprise treatment of a metabolic dysfunction, insulin resistance, type II diabetes, or systemic inflammation or inflammatory disorders. For example, deep subcutaneous adipose tissue may contribute to metabolic dysfunction in people who carry excess amounts of the tissue in their subcutaneous fat layer. Those carrying excess levels of dSAT may experience worsening metabolic health caused by inflammatory signaling of adipocytes on a local and/or systemic level, which may have an associated increase in insulin resistance, glucose intolerance, and type II diabetes. People with metabolic disorders may have associated co-morbidities including obesity, hypertension, dyslipidemia, obstructive sleep apnea, fatty liver disease, and atherosclerosis. For people carrying excessive dSAT and suffer from a metabolic disorder and inflammatory signaling, the selective placement of a slurry into their dSAT layer may result in a reduction of dSAT, and with this, a treatment of metabolic and inflammatory disorders, and/or their associated co-morbidities.

Methods of the invention further comprise treatment of lipedema, lipodystrophy, decrum's disease, lymphedema, lipomatosis, familial multiple lipomatosis, Proteus syndrome, Cowden Syndrome, Modeling disease (benign symmetric lipomatosis), familial angiolipomatosis, lymphatic leakage, de novo adipogenesis, increase in adipocyte cell size, adipocyte proliferation due to excessive leakage of lymphatic including leakage of free fatty acids containing lymph.

A slurry may be administered to a human subject by any suitable method. In some examples, a cannula such as a needle is used to inject the slurry. The needle may be any suitable type of surgical needle. In some examples, the needle is a fenestrated needle. The needle may be a surgical needle of any suitable size. For example, the needle comprises a gauge size of about 8 G to about 25 G.

Methods of the invention may further comprise administering an anesthetic to a treatment area of the subject prior to injecting the slurry. For example, the anesthetic may be a local anesthetic, such as lidocaine.

In some embodiments, the slurry is administered to the subject by injecting the slurry in a pattern. In some embodiments, a plurality of injection sites is used to inject the slurry into the selected treatment site. In certain examples, the amount of slurry injected at each injection site is 2 L or less.

In some embodiments, the sSAT and dSAT are treated at the same time. In an example, a fenestrated needle having a suitable length with fenestrations in both the superficial subcutaneous fat layer and the deep subcutaneous fat layer is used to treat both subcutaneous fat layers at the same time. In an example, the superficial subcutaneous fat layer and the deep subcutaneous fat layer are treated at the same time by injecting a needle and slowly withdrawing the needle, releasing slurry composition in both subcutaneous fat layers.

In certain embodiments, the present invention uses active warming when targeting the selected treatment site. The invention uses active warming of the treatment site that was not selected. For example, if the deep subcutaneous fat layer was selected as the treatment site, active warming may be used on the superficial subcutaneous fat layer. Any suitable method may be used for active warming. For example, active warming may be carried out with a heating source, infrared radiation, radiofrequency, or combination thereof.

In addition, specific areas may be targeted by injection of the slurry by a trained professional in a quick session, or multiple sessions. Because the slurry is injected, patients are not expected to stay in a particular position while undergoing treatments or be subjected to cold temperatures for long periods of time, such as hours. Extensive surgery, long treatment times, and consulting with a plastic surgeon may be avoided.

### BRIEF DESCRIPTION OF THE DRAWINGS

FIG. 1 shows injection of a slurry into sSAT.

FIG. 2 shows injection of a slurry into dSAT.

FIG. 3 shows tissue rewarming of sSAT and dSAT in light of vascular supply.

FIG. 4 shows targeting and removal of sSAT.

FIG. 5 shows targeting and removal of dSAT.

FIG. 6 shows targeting and removal of sSAT and dSAT.

FIG. 7 shows active warming of the sSAT.

FIG. 8 shows active warming of the dSAT.

FIG. 9 shows candidate areas of the body for targeting of sSAT with slurry.

FIG. 10 shows candidate areas of the body for targeting of dSAT with slurry.

FIG. 11 shows variations in fat in the abdomen and lower thigh anatomic areas.

FIG. 12 shows variations in fat by the back and buttocks anatomic areas.

FIG. 13 shows expansion of dSAT and sSAT for men and women.

FIG. 14 shows targeting of multiple sublayers within the sSAT.

FIG. 15 shows delivering ice through multiple fascial compartments.

FIG. 16 shows the multiple layers, sublayers and compartments within the sSAT and dSAT.

FIG. 17 shows fat globules after delivery of slurry.

### DETAILED DESCRIPTION

The invention provides use of slurries for a highly selective, precise, and targeted removal of fat in subcutaneous fat layers, resulting in superior results as compared to conventional methods.

Subcutaneous fat comprises at least a superficial layer and deep layer. The superficial layer of subcutaneous fat is bounded superiorly by the dermis and inferiorly by a fascial layer. The superficial layer provides mechanical support and plays a thermo-insulator and metabolic role. The superficial layer is characterized by a lamellar pattern, consisting of regular, defined cuboid fat lobules tightly organized within vertically oriented fibrous septae. The superficial layer is also highly vascularized as compared to deeper fat layers. However, deep layers are characterized by vessels of a larger lumen size.

The deep, or lower layer of subcutaneous fat, is bounded superiorly by the fascia and inferiorly by muscle. This layer plays a metabolic and inflammatory role in the body. Further, dSAT is a driver of poor aesthetic outcomes, such as excess adiposity. This fat is considered to be distinct from the superficial layer in both form and function. In contrast to the lamellar organization of the superficial layer, deep fat is characterized by a loose areolar pattern. These fat lobules are flat shaped, irregular in size, and are surrounded by high amounts of loose connective tissue.

The differences in mechanical properties between the superficial and deep layers have implications for the activity of injected slurry compositions. It has been shown that the superficial and deep fat layers are distinguishable using standard ultrasound, as the fascial plane is readily visualized. Therefore, one technique in slurry injection to determine which layer is being targeted is to use ultrasound to guide injections to the desired fat layer. Other imaging methods include the use of magnetic resonance imaging (MRI), which also easily distinguishes the two layers. Clinical judgment may be used by using the variations in resistance when the injection needle pierces various layers of tissue to determine needle placement. Furthermore, less injection force may be required for injection into superficial fat as compared to deep fat.

The invention allows for injection into sSAT only, injection into dSAT only, injection into dSAT followed by injection into sSAT, injection into sSAT followed by injection into dSAT, and injection into dSAT and sSAT simultaneously. Therefore, the invention allows for selective targeting of sSAT and dSAT. In some embodiments, injection into the dSAT followed by injection into the sSAT is utilized to allow for visualization of each layer during the injection. Additionally, multiple treatments may be performed, for example with a first session targeting the sSAT and a second session targeting the dSAT. Any layer(s) can be treated in any order in any number of treatments.

To reduce pain associated with injection, methods of the invention may further comprise administering an anesthetic to an area for treatment of the subject prior to injecting the slurry, topically and/or via injection. For example, the anesthetic may be a local anesthetic, such as lidocaine. In certain embodiments, the anesthetic may be administered to a subject a suitable amount of time in advance of the treatment in order to numb the injection area before treatment of the slurry.

The slurry is administered to a human subject by any suitable method. In some examples, the slurry is injected by any suitable means, such as injection by a cannula such as a needle. The needle may be any suitable type of surgical needle. In some examples, the needle is a fenestrated needle. The needle may be a surgical needle of any suitable size. In some examples, the needle comprises a gauge size of about 8 G to about 25 G.

The slurry may also be administered with internal or external pressure on or near the target site to modify administration and/or effect of the slurry. For example, a balloon structure may be deployed at or near a point of delivery to act as an internal pressure device obstructing the flow of blood into a treatment area thus achieving extended cooling after injection. Approaches to delivery of a slurry utilizing balloon structures are disclosed, for example, in International Application Publication No. PCT/US2018/026273; U.S. Patent Application Publication No. 2018-0289538; and U.S. Provisional Application No. 62/482,008, which are incorporated herein by reference in their entirety. In an embodiment, a vasoconstrictor is administered to the subject to reduce blood flow to achieve extended cooling. Pressure may also be applied externally using hand pressure and/or an applicator on the surface of the dermis.

Any suitable amount of slurry that is safe for administering to the human subject may be injected. For example, an amount of slurry administered can be selected based on patient characteristics, the treatment site and/or to produce desired effects of treatment. Treatment with the slurry comprises reduction or removal of fat cells in the human subject by freezing, or cryolipolysis. Treatment may also comprise tightening of skin of the human subject. The tightening of the skin results from a collagen response upon removal and reduction of the fat cells in the subcutaneous fat layer. Reduction of subcutaneous fat may also reduce adipose tissue hypoxia or inflammatory signaling in overweight and obese individuals. Additionally, the slurry may also be utilized to mechanically disrupt fibrous tissue to break up compartments found within the subcutaneous fat, allowing the subcutaneous fat to spread and create a visually smoother appearance, for example in the treatment of cellulite.

Treatment with the slurry may be optimized for cosmetic or aesthetic results, for example to achieve smoothing and to avoid the appearance of sharp edges in the subcutaneous layer or layers. In some embodiments, a profile can be created that correlates to the ice coefficient in the slurry. For example, slurry with a higher ice coefficient can be used to treat the center of a treatment site, while slurry with a lower ice coefficient can be used to treat to the outer perimeter of the treatment site. Any of the slurry properties such as ice coefficient, ice size and ice shape, can be varied to achieve a desired result.

In an embodiment of the present invention, a treatment plan can be created for a subject, for example to determine the slurry properties, volume of slurry to be delivered, and treatment sites such as superficial and/or deep layers. Factors considered in creating a treatment plan for a subject may comprise one or more of gender, height, body weight, body fat percentage, anatomy such as septae rigidity, lifestyle, vitals, medical history, lipid profiles, skin elasticity, medication, nutrition, supplements, demographic, fat saturation, and the like. Fat saturation may be characterized by one or more of imaging, biopsy, and impedance measurement. In embodiments of the present invention, once a plan is created for the subject, the amount of slurry to the administered can be adjusted based on one or more of the area or areas to be treated, the subcutaneous fat layers to be treated, the depth of injection, and the injection pattern to be used.

A computer or artificial intelligence system may be utilized to create a treatment plan for a patient by collecting pre-, peri-, and/or post-injection data from multiple subjects. It is appreciated that the more data points, the more effective the artificial intelligence system will be in creating a treatment plan for a subject. For example, pre-, peri-, and/or post- injection data may be collected for each subject comprising one or more of gender, height, body weight, body fat percentage, the subject's anatomy such as septae rigidity, lifestyle, the subject's vitals, medical history, lipid profiles, skin elasticity, medication, nutrition, supplements, demographic, fat saturation, imaging data, treatment data and fat loss data. Data may be measured by any suitable means. For example, fat loss data may be measured by calipers or any imaging methods such as ultrasound and/or MRI.

In an embodiment, an amount of slurry injected comprises about 2 L or less per injection site. In some examples, the amount of slurry injected is about 1 mL to about 2 L per injection site. Different patients have different amounts of subcutaneous fat. Therefore, some patients may require injection of greater amounts of slurry in order to produce visible effects of reduction and removal of subcutaneous fat. Other patients may require multiple treatments to produce effects of removal or reduction of subcutaneous fat or tightening of the skin as a result of a collagen response.

A slurry for use in the invention may also be injected at multiple treatment sites. For example, the selected treatment sites may be the superficial subcutaneous fat layer, the deep subcutaneous fat layer, or both. For example, the slurry may be injected into the superficial subcutaneous fat layer or deep subcutaneous fat layer at a plurality of injection sites. In some embodiments, the slurry is injected at a plurality of injection sites into both subcutaneous fat layers. In an example, the injection sites may form a pattern, such as a plow, fan, or grid-like pattern, or in a single bolus or multiple bolus injections. In another example, one injection site is used repeatedly, thereby reducing the number of injection sites and concomitant scarring potential. In a plow injection pattern, a single initial target injection site is used followed by a moving needle for additional deposition sites, for example in a linear pattern. In a fan injection pattern, deposition sites form an arc from 1 to 360 degrees. In a bolus injection, the slurry is deposited in a single injection site. The deposition site is where the slurry is deposited, regardless of the injection site, and may be a different site than the injection site or the same site.

The injection pattern can be determined based on the subject's profile, treatment plan, or based on the target site to be treated. For example, an injection pattern and/or volume may be selected to optimize consistency of temperature at the target site. In an embodiment, the injection pattern and/or volume is selected in order to achieve gradient cooling of fat proximate to a target site or injection site. Injection techniques, including the patterns described herein, are known to those of skill in the art.

In some embodiments, the superficial subcutaneous fat layer is treated at the same time as the deep subcutaneous fat layer. For example, the slurry is injected into superficial subcutaneous fat, and then the needle is moved deeper in the deep subcutaneous fat regions. In an embodiment, a fenestrated needle having a suitable length with fenestrations in both the first subcutaneous fat layer and the second subcutaneous fat layer is used to treat the first and second subcutaneous fat layers at the same time. In another example, the superficial subcutaneous fat layer and the deep subcutaneous fat layer are treated at the same time by injecting a needle and slowly withdrawing the needle, releasing slurry in both subcutaneous fat layers.

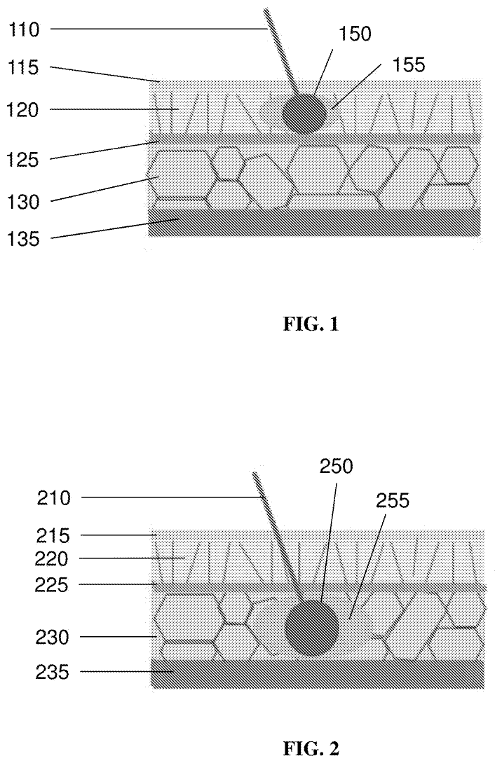

FIG. 1 shows injection of a slurry into superficial fat (sSAT) 120. The core of ice crystals 150 is at the injection site with a fluid component 155 of the slurry expanding from the injection needle 110. The sSAT 120 is bounded superiorly by the skin 115 and inferiorly by the fascia 125. The deep fat layer (dSAT) 130 is bounded superiorly by the fascia 125 and inferiorly by muscle 135. Similarly, FIG. 2 shows injection of a slurry into dSAT 230. The core of ice crystals 250 is at the injection site with a fluid component 255 of the slurry expanding from the injection needle 210. The sSAT 220 is bounded superiorly by the skin 215 and inferiorly by the fascia 225. The dSAT 230 is bounded superiorly by the fascia 225 and inferiorly by muscle 235.

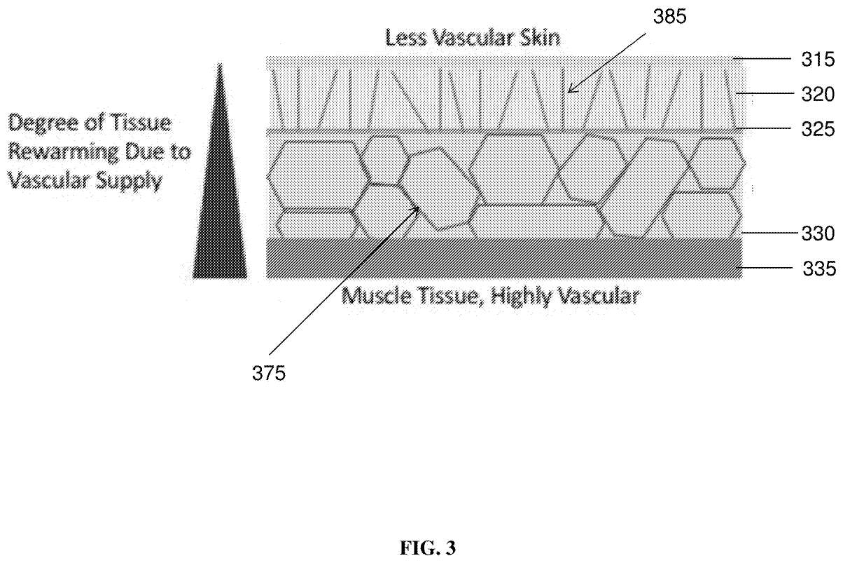

In another example, in order to obtain equivalent cooling durations, it may be necessary to inject a greater volume into the dSAT compared to the sSAT. Given the increased radially spread of a fixed volume in the dSAT, the concentration of ice may be less dense and melt more quickly than the more densely packed ice in the sSAT. Additionally, dSAT is closer to highly vascular underlying muscle. The proximity to underlying muscle may cause the area to rewarm more quickly than the less vascular sSAT that is further from muscle. FIG. 3 shows the degree of tissue rewarming due to vascular supply which decreases when traveling upwards from the highly vascular muscle tissue towards the less vascular skin. In relation, dSAT has a greater relative vascularity than sSAT.

The lamellar pattern of the sSAT is shown in FIG. 3, as well as the loose areolar pattern of dSAT. The sSAT 320 consists of regular, defined cuboid fat lobules tightly organized within vertically oriented fibrous septae 385. The dSAT 330 has a loose areolar pattern with fat lobules 375 that are flat shaped, irregular in size, and are surrounded by high amounts of loose connective tissue. The skin 315, fascia 325, and muscle 335 are also designated.

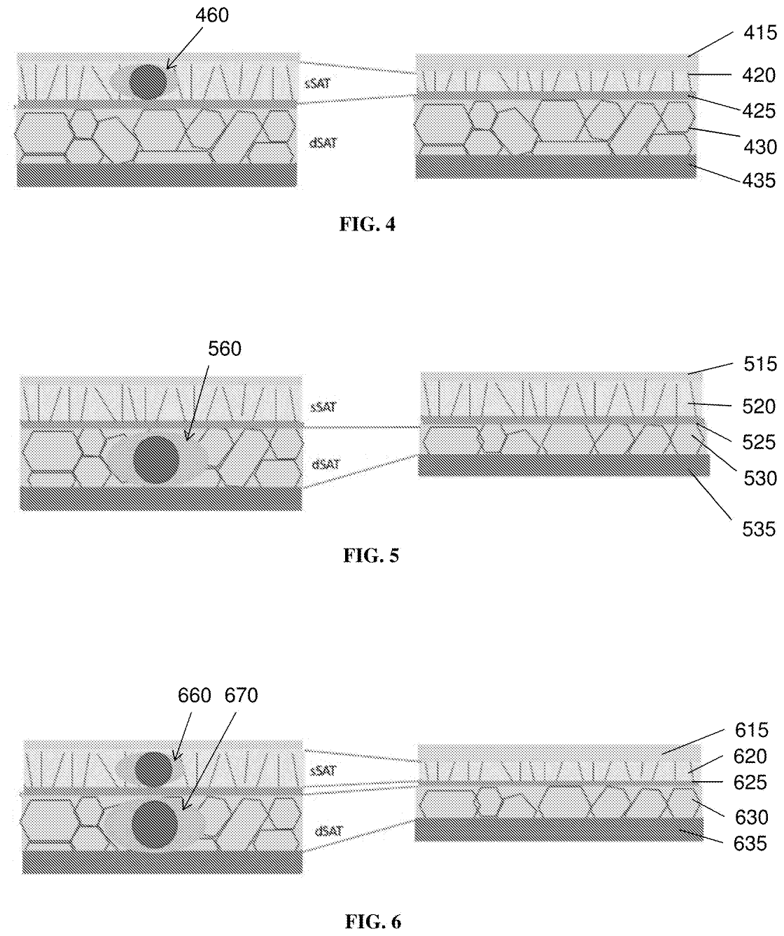

In another example, shown in FIG. 4, sSAT can be selectively targeted with a slurry 460 to target and remove sSAT. The slurry 460 is injected in the sSAT 420 located below the skin 415 and above the fascia 425. The dSAT 430 is shown between the fascia 425 and the muscle 435.

In another example, shown in FIG. 5, dSAT can be selectively targeted with a slurry 560 to target and remove dSAT. The slurry 560 is injected in the dSAT 530 located between the muscle 535 and the fascia 525. The dSAT 530 sits below the sSAT 520, which is located between the fascia 525 and the skin 515.

In another example, shown in FIG. 6, both sSAT and dSAT can be selectively targeted with slurries 660 and 670 to target and remove both dSAT and sSAT. The slurry 660 is injected in the sSAT 620 located below the skin 615 and above the fascia 625. The slurry 670 is injected in the dSAT 630 located between the muscle 635 and the fascia 625. Slurry 660 and slurry 670 may be the same slurry or may be different slurries (slurry compositions and properties are described below).

Targeting of the respective subcutaneous fat layer occurs by localizing slurry injections to that layer (or sublayer or compartments within a layer). Further, reducing or removing the fat cells may result in a collagen response, such as shown by thickening of the skin in FIG. 4 and FIG. 6. The collagen response works to tighten the skin in the treated area.

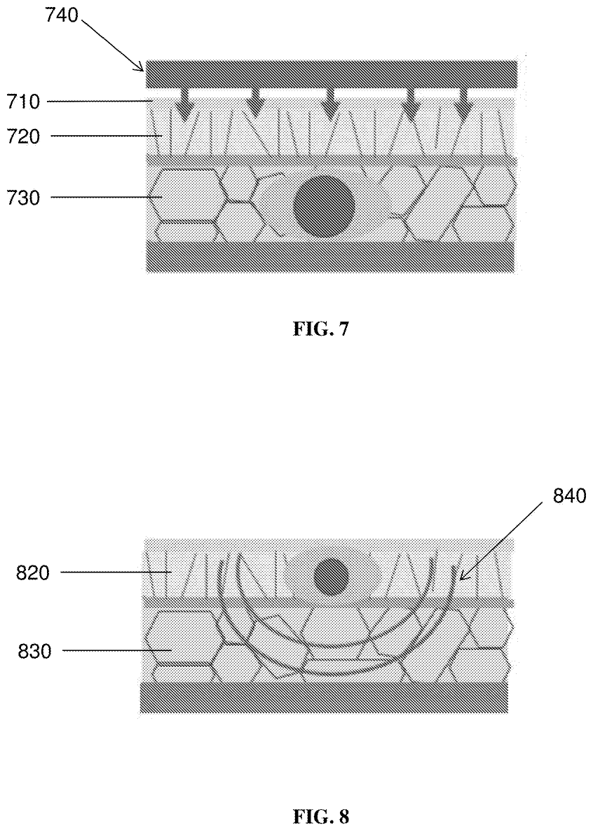

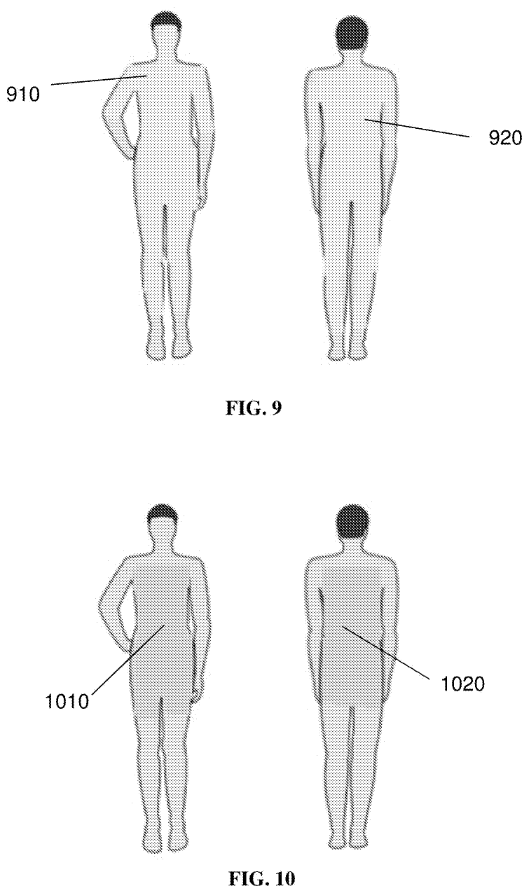

In addition to achieving selectivity by localizing slurry injection, selectivity can be achieved or augmented by combining an injection with active warming of the layers that are not targeted. For example, as shown in FIG. 7, to limit slurry cooling to dSAT 730, sSAT 720 could be actively warmed through application of a heating source 740 to the skin 710 or use of infrared radiation. Conversely, as shown in FIG. 8, to selectively target sSAT 820, the dSAT 830 could be actively warmed using a modality such as radiofrequency 840.

Given the anatomy of the fat layers, not all body areas containing excess unwanted subcutaneous fat are suitable for treatment of sSAT and dSAT. The body areas that contain dSAT also contain a sSAT layer, making these areas suitable for both depths of treatment, whereas many body areas only have a sSAT layer. For example, aesthetic removal of submental, upper arm, lower thigh medially or laterally, supragenicular, ankle, or facial fat only contain sSAT, whereas other areas of removal are suitable to both dSAT and sSAT targeting, such as abdomen, flank, lumbar, or buttock fat.

FIG. 9 shows candidate areas of the body for targeting of sSAT with slurry. Candidate areas on the front of the body are designated by shaded area 910, while candidate areas on the back of the body are designated by shaded area 920.

FIG. 10 shows candidate areas of the body for targeting of dSAT with slurry. Candidate areas on the front of the body are designated by shaded area 1010, while candidate areas on the back of the body are designated by shaded area 1020.

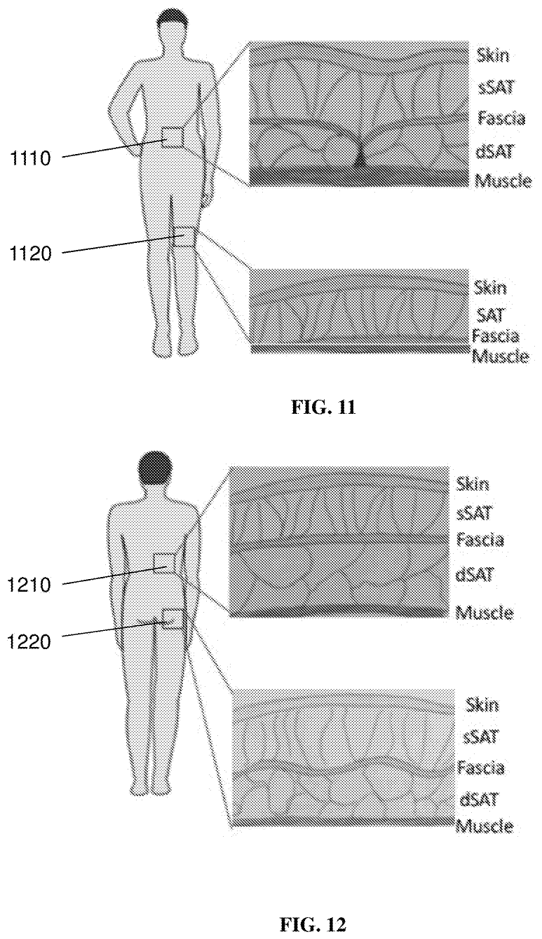

FIG. 11 shows variations in fat by anatomic area, particularly the abdomen 1110 and lower thigh 1120 areas. FIG. 12 shows variations in fat by anatomic area, particularly the back and buttocks.

In addition to the differences in the mechanical properties of tissue, there are molecular, genetic and functional differences as well. Magnetic resonance imaging has shown that dSAT contains more saturated fatty acids compared to sSAT. It is also widely known that degree of saturation of fat affects the temperatures at which phase changes occurs, with greater saturation correlating to higher freezing points. Hence, in the context of slurry injection less cooling will be required to induce cryolipolysis in dSAT relative to sSAT. For example, for equivalent volumes of sSAT and dSAT, successful cryolipolysis to target and remove that volume would require less ice for the dSAT compared to the sSAT. Ice volume can be adjusted either through changing injection volume and/or ice coefficient. The ice coefficient is the percentage of ice in the slurry, and in some embodiments, the ice coefficient of the slurry may range from 2-70%.

Further, there are molecular differences between sSAT and dSAT. A study demonstrated that sSAT preferentially expressed metabolic genes such as adiponectin (ADIPOQ), adiponectin receptor 2 (ADIPOR2) and caveolin 2 (CAV2) compared to dSAT. sSAT also preferentially expressed serum amyloid protein genes SAA1, SAA2, and SAA4. In contrast, dSAT preferentially expressed leptin receptor gene (LEPR), apolipoprotein C1 (APOC1), adrenergic alpha 1B receptor (ADRAB1), adenosine A2a receptor (ADORA2A), interleuking 1 receptor antagonist (IL1RN), which indicates more of an inflammatory tissue profile. Additional research showed that other genes of inflammation such as MCP1 and IL6 were preferentially expressed in dSAT in men, but not women. This research provides further evidence that sSAT and dSAT are distinct layers of adipose tissue not only in terms of structure, but in terms of gene expression and function. Hence, cryolipolysis can be successfully performed on a distinct type of adipose tissue from the sSAT typically targeted using cold temperature.

These genetic differences also have implications for the use of cryolipolysis for therapeutic applications outside of aesthetic fat removal. In one example, for patients with or at risk of kidney disease, slurry treatment of sSAT may be of interest, given sSAT's preferential expression of serum amyloid proteins, which are common in kidney disease. In contrast, sSAT may want to be preserved in patients with or at risk of metabolic disorders such as insulin resistance or type II diabetes, as sSAT has been shown to be a metabolically protective layer of fat in patients with type II diabetes.

One clinical application of injection of slurry into SAT is for selective targeting and removal of dSAT. Recent research has demonstrated that pro-inflammatory dSAT may play a key role in metabolic dysfunction, potentially equivalent to that of visceral adipose tissue (VAT), a fat depot with well-established connections to metabolic dysfunction and type II diabetes.

Studies have shown that a disproportional accumulation of VAT correlates with insulin resistance as well as common associated metabolic diseases. Examples of metabolic diseases include hypertension, hyperlipidemia, elevated triglycerides, and non-alcoholic steatohepatitis. This association is particularly marked in males.

In certain embodiments, methods of the invention further comprise treatment of a metabolic dysfunction, insulin resistance, type II diabetes, or systemic inflammation or inflammatory disorders. For example, deep subcutaneous adipose tissue may contribute to metabolic dysfunction in people who carry excess amounts of the tissue in their subcutaneous fat layer. Those carrying excess levels of dSAT may experience worsening metabolic health caused by inflammatory signaling of adipocytes on a local and/or systemic level, which may have an associated increase in insulin resistance, glucose intolerance, and type II diabetes. People with metabolic disorders may have associated co-morbidities including obesity, hypertension, dyslipidemia, obstructive sleep apnea, fatty liver disease, and atherosclerosis. For people carrying excessive dSAT and suffer from a metabolic disorder and inflammatory signaling, the selective placement of a slurry into their dSAT layer may result in a reduction of dSAT, and with this, a treatment of metabolic and inflammatory disorders, and/or their associated co-morbidities.

Accordingly, in embodiments of the present invention, it may be desirable to target only one layer of subcutaneous fat from either of the sSAT and dSAT. Treatment of dSAT alone may result in a desired metabolic response whereas treatment of sSAT alone may result in a desired cosmetic or aesthetic effect.

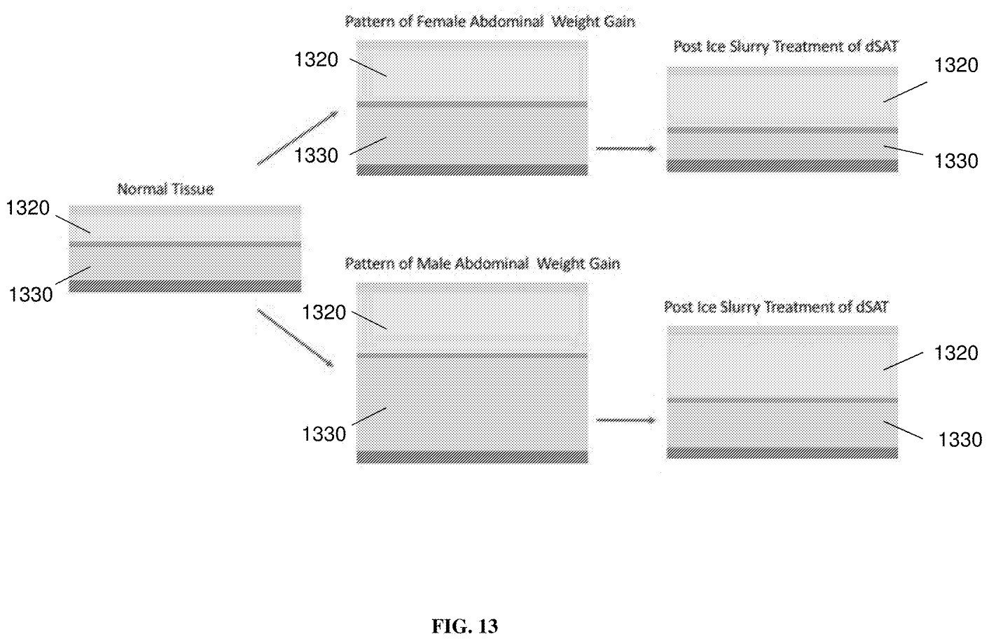

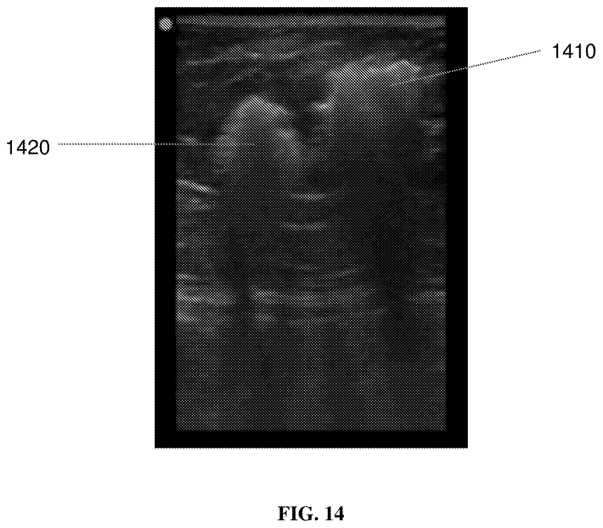

FIG. 13 shows that increased adiposity in men is characterized by disproportionate expansion of dSAT 1330, whereas in women sSAT 1320 and dSAT 1330 tend to expand proportionately. Such observations are consistent with the observation that dSAT is the strongest predictor of peripheral and hepatic insulin resistance in men, independent of other adipose indices. Hence, selective targeting and removal of dSAT using slurry may be of disease modifying value for metabolic conditions, especially in the male population.

FIG. 14 shows that the slurry enables precise targeting of sublayers within superficial subcutaneous fat with slurries 1410 and 1420. The slurry 1410 is injected within the superficial subcutaneous layers and further within a sublayer of fat close to the skin. The slurry 1420 is injected within a sublayer deeper within the sSAT. Selective targeting is suitable for cryolipolysis when targeting and removing multiple sublayers within the subcutaneous fat. Slurry 1410 and slurry 1420 may be the same or different slurry.

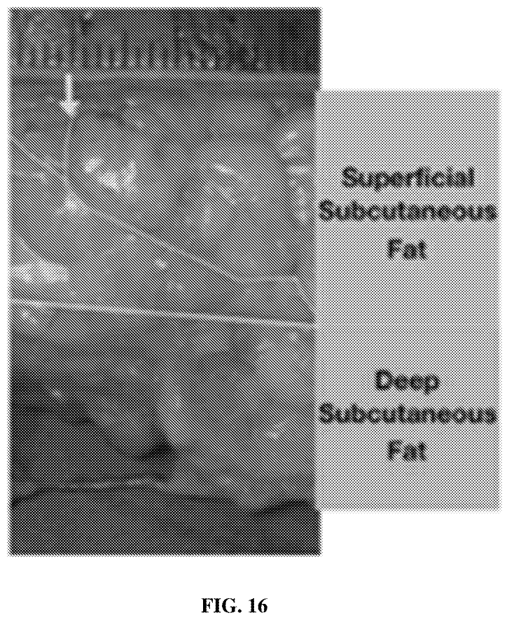

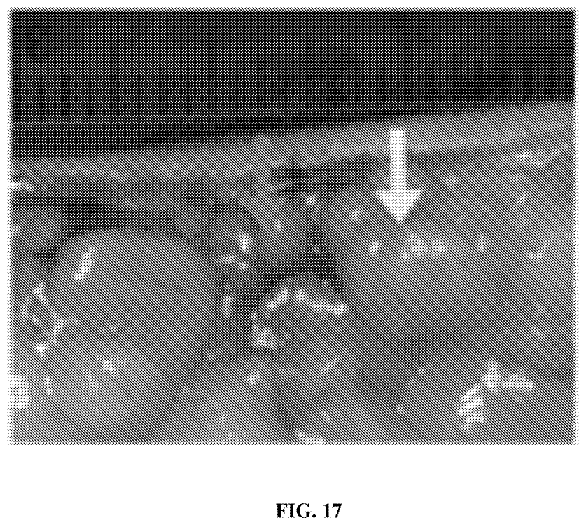

As shown in FIG. 16, the slurry enables highly precise targeting of subcutaneous fat. The slurry can be used to target the superficial or deep subcutaneous fat. It can also be used to target compartments within the superficial subcutaneous fat. In an excised specimen of human subcutaneous fat from the abdomen, a fascial plane within the superficial subcutaneous fat is highlighted gray. The slurry is injected superior to this compartment or inferior to this compartment depending on which area is desired to be removed. Ice slurry can be used for even more precise delivery, to selectively target and remove individual fat globules within the superficial subcutaneous fat. In FIG. 17 precise delivery of ice slurry reduced the fat globule(s) indicated by the blue arrow, while leaving adjacent globules indicated by the yellow arrow unaffected.

The slurry may also be utilized to mechanically disrupt fibrous tissue to break up compartments found within the subcutaneous fat, allowing the subcutaneous fat to spread and create a visually smoother appearance, for example in the treatment of cellulite. The slurry creates enough force to mechanically dissect fibrous tissue strands. In a preferred embodiment, the slurry creates enough force without an injection assistance device to mechanically dissect fibrous tissue strands.

Pre- or post-injection steps may also be utilized to optimize slurry treatment results. For example, a massaging step may be utilized to increase fat cell damage and/or the mechanical force of the ice in the slurry. In an embodiment, the massaging is performed to puncture one or more cell membranes. The massaging step may be used to position or shape the slurry post injection. Massaging can be performed by any mechanical means, for example by hand, vibration, an applicator, or by acoustic means. Imaging pre-injection can be utilized to create a treatment plan and may further be used to develop the profile for the subject. For example, the septae of the subject may be damaged prior to injection of the slurry to allow the slurry to flow more smoothly. In an embodiment the septae in damaged by puncture. In another embodiment the septae is damaged by massaging.

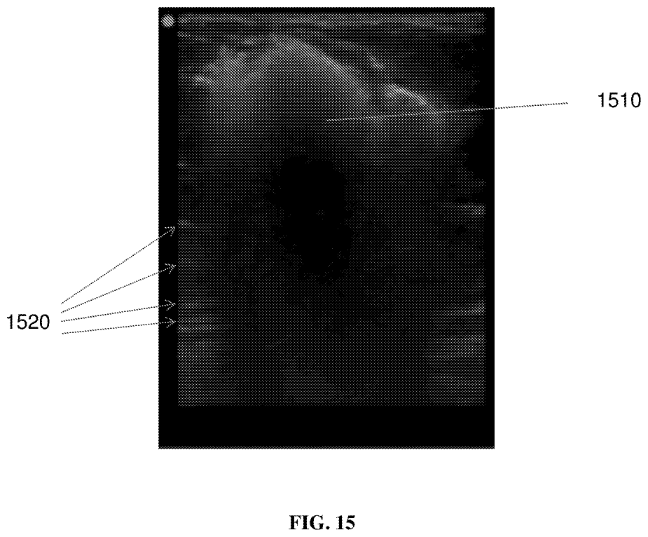

FIG. 15 shows a single slurry injection 1510 within the subcutaneous fat positioned using the force of the clinician's hand to dissect through multiple compartments 1520 within the fat layer. This injection technique is suitable for cryolipolysis of the subcutaneous fat and is optimal for cosmetic treatments where breaking and remodeling of fibrous tissue between fat layers is desired, such as treatment of cellulite. Disruption of the fibrous tissue can be accomplished or modified based on the amount of slurry administered and the ice content of the slurry.

As an example, the slurry may be administered to target a large area, such as the abdomen utilizing a large volume of slurry and/or a slurry with a high ice content, for example as 20% or 70%. In another example, the slurry may be administered to target a small area, such as the chin, utilizing a smaller volume of slurry and/or a slurry with a low ice content, such as 2%.

In certain embodiments, methods of the invention further comprise treatment of lipedema, lipodystrophy, decrum's disease, lymphedema, lipomatosis, familial multiple lipomatosis, Proteus syndrome, Cowden Syndrome, Modeling disease (benign symmetric lipomatosis), familial angiolipomatosis, lymphatic leakage, de novo adipogenesis, increase in adipocyte cell size, adipocyte proliferation due to excessive leakage of lymphatic including leakage of free fatty acids containing lymph.

For example, lipedema is a disorder of the adipose tissue that is associated with abnormal deposition of subcutaneous fat that is strongly associated with accompanying lymphatic and vascular dysfunction in the affected areas. Lipedema is almost exclusively observed in women. The abnormal deposition of fatty tissue predominantly affects the lower limbs, bilaterally, and is associated with pain and tenderness in the large areas. Lipedema is a distinct condition in its pathophysiology and presentation from obesity and lymphedema. Recent work has classified lipedema into two subcategories of aberrant fat deposition-columnar or lobar, either of which are suitable for treatment using ice slurry. At present the mainstay of treatment is liposuction, preformed either tumescently or with laser-assistance to de-bulk the fatty tissues. However, given the large treatment areas and associated morbidity of surgical treatment by liposuction, there is an urgent need for alternative therapies that are area selective for fat and can treat diverse geometries, areas, and depots of fatty tissue, making the slurry an excellent alternative option for treatment. See Okhavat et al.(2015) Int. J. Low Extrem. Wounds 14(3):262-7. Accordingly, in embodiments of the present invention the slurry may be administered to selectively target and remove subcutaneous fat in the lower limbs.

Decrum's disease is a rare disorder characterized by multiple painful subcutaneous growths of adipose tissue. Decrum's disease is characterized into four subtypes: generalized diffuse, generalized nodular, localized nodular and juxta-articular. The slurry may be used to reduce the painful fatty growths in any of these subtypes and relieve pain. See Hansson et al. (2012) Orphanet J Rare Dis. 7:23.

Slurry may also be used to improve symptoms of lymphedema, especially lymphedema secondary to the excision of lymph nodes. Following surgical excision of lymph nodes, often performed in the context of surgical management of malignancies, lymph accumulates distal to the excised area and is associated with the proliferation of adipose tissue in that area. At present, liposuction is a surgical option for advanced lymphedema, but remains an invasive option requiring general anesthesia and prolonged recovery time. Hence, slurry injection may present a minimally invasive, non-surgical option for reducing limb volume and/or improving limb function and/or reductions in extracellular fluid as assessed by bioimpedance spectroscopy. See Boyages et al. (2015) Ann Surg Oncol. 22:Suppl 3:S1263-70.

In certain embodiments, methods of the invention further comprise treatment of lipomatosis, aberrant fat tissue proliferation, and/or lipomas associated with genetic syndromes such as familial multiple lipomatosis, Proteus syndrome, Cowden Syndrome, Modeling disease (benign symmetric lipomatosis), and familial angiolipomatosis.

In additional embodiments, methods of the invention further comprise treatment of lymphatic leakage, for example de novo adipogenesis, increase in adipocyte cell size, and/or adipocyte proliferation due to excessive leakage of lymphatic, which may include leakage of free fatty acids containing lymph. See Escobedo et al. (2017). Cell MEtab. 26(4):598-609.

Slurries for use in the invention may be any suitable composition capable of removing adipose cells and subcutaneous fat. Slurry compositions are described in U.S. Provisional Patent Application Ser. No. 62/741,279 which is incorporated by reference in its entirety herein. Slurry can be generated from a solution using any of the slurry generation systems and methods described in U.S. Provisional Patent Application Ser. No. 62/743,830 which is incorporated by reference in its entirety herein.

Slurry compositions of the present invention may comprise a solvent and one or more additives. The solvent may be any solvent suitable for injection. In certain embodiments, the solvent is liquid water. In some embodiments, additives can be chosen that have a low molecular weight, therefore affect certain properties while minimizing impact on other properties. For example, including more additives improves the flowability and ice particle size, but also increases osmolarity and makes the solution more hypertonic.

In some embodiments, additives are inactive ingredients. Any suitable additive may be added to the solution or the slurry, including any substance on the FDA GRAS list, which is incorporated herein in its entirety.

In some embodiments, the additives comprise one or more of a salt, a sugar, and a thickener. In an embodiment, the salt is NaCl at about 2.25% by mass or lower. In an embodiment, the sugar is glycerol at about 2% by mass or lower. In an embodiment, the thickener is CMC or Xanthan Gum at about 0.75% by mass or lower. Additional additives may be included to affect various properties of the slurry.

Any acceptable or suitable concentration of one or more additives may be used in the present invention and may be selected based on the treatment. For example, for intradermal, subcutaneous, or intramuscular routes of administration, additives include sodium chloride (saline), glycerin/glycerol, dextrose, sodium CMC, xanthan gum, and polyethylene glycol. For example, acceptable concentrations of sodium chloride (saline) are about 0.9% for soft tissue use and about 2.25% for subcutaneous use, while acceptable concentrations of glycerin/glycerol are about 1.6% to about 2.0% for dermal use and about 15% for subcutaneous use. Further, acceptable concentrations of dextrose are about 5% w/v for intramuscular use and about 7.5% per unit dose for intramuscular-subcutaneous use. For example, acceptable concentrations of sodium CMC are about 0.75% for intralesional use, about 3% for intramuscular use, and about 0.5% to about 0.75% for soft tissue use. As another example, acceptable concentrations of xanthan gum are about 1% for intra-articular use in animal studies and about 0.6% for FDA ophthalmic use. Further, acceptable concentrations of polyethylene glycol, such as Polyethylene Glycol 3350, are about 2.0% to about 3.0% for FDA soft tissue use and about 4.42% for subcutaneous use.

In some embodiments, the salt is saline, a solution of sodium chloride (NaCl) in water. Other examples of salts include potassium, calcium, magnesium, hydrogen phosphate, hydrogen carbonate. In some embodiments, glycerol is an additive. In some embodiments, dextrose is an additive. In some embodiments, additives for affecting the viscosity include CMC and Xanthan Gum. In some embodiments, an additive may comprise a buffer to stabilize the pH. In some embodiments, the solution pH is about 4.5 to about 9. In some embodiments, an additive may comprise an emulsifier to create a smooth texture. In some embodiments, an additive may comprise a nanoparticle, for example, TiO2. In some embodiments, an additive may comprise an agent configured as coating for the ice crystals which may prevent agglomeration after formation may be included. In some embodiments, an additive may comprise IVF Synthetic Colloids at amounts of about 6.0% Hetastarch in about 0.9% sodium chloride; Poloxamer 188 at amounts of about 0.2% subcutaneous; Propylene Glycol at amounts of about 0.47% to about 1.4%; Benzyl Alcohol at amounts of FDA about 0.9% to about 1.4%; gelatin at amounts of FDA subcutaneous about 16%; and Icodextrin used frequently in peritoneal dialysis at amounts of about 7.5%.

In certain embodiments, the slurry and solution compositions have an osmolarity lower than about 2,200 mOsm/L. In some embodiments, the osmolarity is less than about 600 mOsm/L. In such an embodiment, the slurry may comprise about 0.9% saline; about 1.0% to about 2.0% dextrose; about 1.0% to about 1.6% glycerol; less than about 0.5% sodium CMC; and less than about 0.6% xanthan gum. In one embodiment, the slurry composition may be about 500 mOsm/kg to about 700 mOsm/kg and comprise about 0.9% to about 1.4% saline; about 2.0% to about 4.0% dextrose; about 1.7% to about 2.0% glycerol; about 0.6% to about 1.0% sodium CMC; and about 0.6% to about 1.0% xanthan gum. In another embodiment, the slurry composition may be about 700 mOsm/kg to about 900 mOsm/kg and comprise about 1.5% to about 1.7% saline; about 5.0% to about 7.5% dextrose; about 3.0% to about 5.0% glycerol; about 1.0% to about 3.0% sodium CMC; and about 1.0% xanthan gum. In some embodiments, the slurry composition may be greater than about 1,000 mOsm/kg. In such an embodiment, the slurry may comprise about 1.8% to about 3.0% saline; about 10% dextrose; greater than about 5.0% glycerol; sodium CMC; and xanthan gum. Additives can be selected and included in any concentration suitable to generate a slurry have certain characteristics, for example to increase or decrease the osmolality.

Once the slurry solution is injected into a subject's body, the slurry causes cryolipolysis, or cell death by freezing of fat cells. Targeted removal of subcutaneous fat is possible using the injected slurry if a temperature of the slurry is cold enough to freeze fat cells and cause cell death. After cell death, the body naturally processes and eliminates the dead fat cells. In particular, the slurry has a high percentage of ice that acts to kill the adipocyte cells in the sSAT and dSAT by freezing the adipocytes. For example, the percentage of ice particles in the slurry is in a range of about 2% to about 70%. To kill the adipocytes, or fat cells, in the subcutaneous fat layers, the slurry injected into the sSAT and dSAT should have a cold temperature. However, the temperature should be warm enough to avoid tissue redness, blistering, tissue necrosis, and ulceration. In some embodiments, the temperature is about −25° C. to about 10° C. In some embodiments, the temperature is about −6° C. to about 0° C.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A method comprising:
injecting a slurry into a subject at a treatment site selected from the group consisting of (i) a deep subcutaneous fat layer, (ii) a superficial subcutaneous fat layer, and (iii) the deep subcutaneous fat layer and the superficial subcutaneous fat layer; thereby inducing cryolipolysis in the selected treatment site by the slurry.

2. The method of claim 1, wherein the slurry is injected into
(i) the deep subcutaneous fat layer only,
(ii) the superficial subcutaneous fat layer only,
(iii) the deep subcutaneous fat layer followed by the superficial subcutaneous fat layer,
(iv) the superficial subcutaneous fat layer followed by the deep subcutaneous fat layer, or
(v) the deep subcutaneous fat layer and the superficial subcutaneous fat layer simultaneously.

3. The method of claim 1, wherein the slurry is injected into the deep subcutaneous fat layer only.

4. The method of claim 1, wherein the treatment site comprises a plurality of injection sites.

5. The method of claim 4, wherein the plurality of injection sites form a pattern.

6. The method of claim 5, wherein the pattern is a plow pattern, a grid pattern, a fan pattern, a single bolus injection, or a multiple bolus injection.

7. The method of claim 4, wherein 2 L or less of slurry is injected per injection site.

8. The method of claim 1, wherein a cannula is used to inject the slurry.

9. The method of claim 8, wherein the cannula comprises a needle comprising a gauge size of about 8 G to about 25 G.

10. The method of claim 9, wherein the needle comprises a fenestrated needle.

11. The method of claim 1, wherein the method further comprises tightening of tissue of the subject.

12. The method of claim 1, wherein the method further comprises disrupting fibrous tissue within the treatment site or sites.

13. The method of claim 12, wherein the method comprises inducing cryolipolysis in a plurality of sublayers and/or compartments within the selected treatment site by the slurry.

14. The method of claim 1, wherein the method comprises inducing cryolipolysis in one or more sublayers and/or compartments within the selected treatment site by the slurry.

15. The method of claim 1, wherein the method further comprises treatment of a metabolic dysfunction, insulin resistance, type II diabetes, or systemic inflammation or inflammatory disorders.

16. The method of claim 1, wherein the method treats excess fat, obesity, and loose skin.

17. The method of claim 1, wherein the method treats lipedema, lipodystrophy, decrum's disease, lymphedema, lipomatosis, familial multiple lipomatosis, Proteus syndrome, Cowden Syndrome, Modeling disease (benign symmetric lipomatosis), familial angiolipomatosis, lymphatic leakage, de novo adipogenesis, increase in adipocyte cell size, adipocyte proliferation due to excessive leakage of lymphatic including leakage of free fatty acids containing lymph.

18. The method of claim 1, wherein the method further comprises administering an anesthetic to the subject prior to injecting the slurry.

19. The method of claim 18, wherein the anesthetic is a topical anesthetic.

20. The method of claim 1, wherein the method does not comprise administering an anesthetic to the subject prior to injecting the slurry.

21. The method of claim 1, wherein the method further comprises applying pressure to the treatment site.

22. The method of claim 12, wherein the method further comprises applying pressure to the treatment site.

23. The method of claim 1, wherein the slurry has a temperature of about −25° C. to about 10° C.

24. The method of claim 1, wherein the slurry comprises ice particles.

25. The method of claim 24, wherein a percentage of ice particles in the slurry comprises about 2% to about 70%.

26. The method of claim 1, wherein the method further comprises active warming of a treatment site that was not selected.

27. The method of claim 26, wherein active warming is carried out with a heating source, infrared radiation, radiofrequency, or combination thereof.

28. A method for selective disruption of one or more subcutaneous fat layers comprising injecting a slurry at a temperature of about −25° C. to about 10° C. into a subject at a treatment site proximate to one or more subcutaneous fat layers, wherein injection of the slurry reduces the temperature of the one or more subcutaneous fat layers proximate to the treatment site to induce cryolipolysis by the slurry.

29. The method of claim 1, further comprising creating a profile for the subject.

30. The method of claim 1, further comprising creating a treatment plan for the subject.
